(19) United States
(12) Patent Application Publication
(10) Pub. No.: US 2004/0116862 A1
(43) Pub. Date: Jun. 17, 2004

(54) Programmable cassette pump for injecting medicaments

(76) Inventor: Claude Ray, Montezillon (CH)

Correspondence Address: VAN TASSEL AND ASSOCIATES, POST OFFICE BOX 2928, BELLAIRE, TX, 77402-2928, US

(21) Appl. No.: 10/474,895

(22) PCT Filed: Apr. 4, 2002
(86) PCT No.: PCT/CH02/00190

(30) Foreign Application Priority Data
Apr. 10, 2001 (EP) 01810354.9

(51) Int. Cl. A61M 1/00; G05B 15/00

(52) U.S. Cl. 604/151; 700/1

(57) ABSTRACT

The invention relates to a programmable pump for injecting medicaments, comprising two parts which can be detachably coupled i.e. a cassette (12) containing a liquid to be injected, and a pump module provided with actuating means so that the liquid can exit from the cassette. The cassette (12) comprises an electronic memory (66) wherein data relating to the prescribed injection program has been previously stored. The invention is also characterized in that when the two parts are coupled, said actuating means are electrically connected to the memory and react to data contained therein in order to inject the liquid according to the desired program.

The present invention concerns miniaturized pumps for injecting medicaments according to a prescribed program. It relates, more particularly, to a programmable medical pump made up of a pump module and of a cassette containing the medicament, which couple in a detachable manner.

The invention also relates to equipment intended for the programming of such a pump.

Miniaturized pumps for medical use have been known for several years. Light and small, they are carried by the patient in a discreet and comfortable manner and allow him or her to administer, subcutaneously or intravenously, and continuously or according to a specified program, controlled quantities of medicinal solutions, without, however, having to be confined to bed and linked to a bulky, expensive and noisy apparatus.

Such pumps are, often, of rotary peristaltic type. The principle thereof consists in employing a deformable plastic conduit linked to a reservoir containing the medicinal solution and in squeezing it locally against a bearing piece of rounded shape by means of idle rollers mounted on a rotor driven by a motor acting via a gear train. The liquid is thus sucked from the reservoir and pushed toward the outlet so as to be injected into the body of the patient.

Patent FR 2 753 235 and document EP 01810096.6, for example, describe cassette pumps of this type.

During the design of these pumps, it is particularly important to be mindful of their ease of programming. Intended to be used in a non-hospital environment, it must, in fact, be possible for them to be operated safely by any carer, or even by the patient him or herself, without their having received special training or having to read lengthy and complicated directions for use. However, these user-friendliness and safety requirements are rarely satisfied by pumps currently on the market. There is then a risk of their remaining a hospital equipment reserved for a few nurses, thus robbing them of a large part of the benefit they offer.

The aim of the present invention is therefore to provide a cassette pump that can be used immediately by anyone, without requiring any training or special knowledge.

More precisely, the invention relates to a programmable pump for injecting medicaments, comprising two parts that can be coupled in a detachable manner, i.e.:

a cassette containing a liquid to be injected, and

a pump module fitted with actuation means for discharging the liquid from the cassette to the exterior,

characterized in that the cassette comprises an electronic memory in which have previously been recorded data relating to the prescribed injection program and in that, when the two parts are coupled, the actuation means are electrically connected to the memory and respond directly to the data that it contains so as to inject the liquid according to the desired program.

Admittedly, patent U.S. Pat. No. 5,920,054 describes a system for administering a liquid to a patient, in which a module of fluid prepared in the laboratory is coupled to an injection module at the administration site. The module of fluid comprises a memory containing information such as the type, the concentration and the volume of the fluid, and information relating to the calibration of the injection module. The latter uses the information received to calculate its own operating parameters.

It will be noted, however, that the aforesaid U.S. document does not in any way suggest the principle, which is the main characteristic of the present invention:

of associating data relating to the administration program with the cassette containing the liquid to be injected, and

without requesting any calculation by the pump module actuation means, of placing the latter under the direct orders of the cassette so as to execute the program contained in its memory.

This automatic programming of the pump module by the cassette at the moment of their coupling constitutes an essential advantage of the pump according to the present invention since its use on the patient is of the greatest simplicity.

Preferably, the pump module is of peristaltic type. It comprises a casing and, inside the latter, a rotor furnished with idle rollers, a stepper motor for driving the rotor and a microprocessor for the control of the motor.

In this case, the cassette advantageously comprises a casing in which the memory is fixed and, placed in a detachable manner inside this casing, a medicament module comprising a pouch intended to contain the liquid to be injected, a flexible conduit linking the pouch to the exterior and a bearing piece against which, during the rotation of the rotor, the rollers of the latter will, upon each revolution, squeeze the conduit and thus push out, by means of peristaltic motion, the liquid contained in the pouch.

According to the above embodiment, the two casings comprise members for coupling one to the other and are fitted with conducting tracks, which, upon the coupling of the casings, are placed in communication in such a way as to interconnect the memory and the microprocessor.

Advantageously, the data recorded in the memory comprise, for each step of the program corresponding to a constant injection rate, the start and end instants of the step as well as the number of pulses applied to the motor per unit time, determining the speed of rotation of the rotor and hence the injection rate. The data recorded in the memory comprise, in addition, for each bolus requested by the patient, its duration, the number of additional driving pulses per unit time, the number of boluses authorized per day and their minimum interval.

The present invention also relates to equipment for programming the pump defined hereinabove, characterized in that it comprises:

a computer containing, in memory, for a plurality of medicaments likely to be administered by the pump, the maximum permitted instantaneous injection rate, the maximum permitted daily injectable quantity,

fixed on the pouch, a label for identifying the medicament that it contains,

means for reading the label, which are linked to the computer, and

a data exchange interface, linked to the computer and designed to link it to the memory of the cassette.

According to a preferred mode of implementation, the computer is programmed to perform, on the basis of the information delivered by the label and in response to the instructions supplied by an operator in respect of the desired injection program:

verification that said instructions comply with the permitted values contained in memory,

calculation, for each step of the program corresponding to a constant injection rate, of the start and end instants of the step as well as of the number of pulses applied to the motor per unit time, then

at the request of the operator, transfer of the calculated values to the memory of the cassette.

Advantageously the computer contains, in addition, for certain, at least, of the medicaments likely to be administered by the pump, the number of boluses authorized per day, the maximum volume of each bolus and the minimum space between two boluses. In this case, the computer is programmed to perform, in addition, the calculation, for each bolus requested by the patient, of its duration and of the number of additional driving pulses per unit time.

Other characteristics of the invention will become apparent from the description that follows, given with regard to the appended drawing, in which:

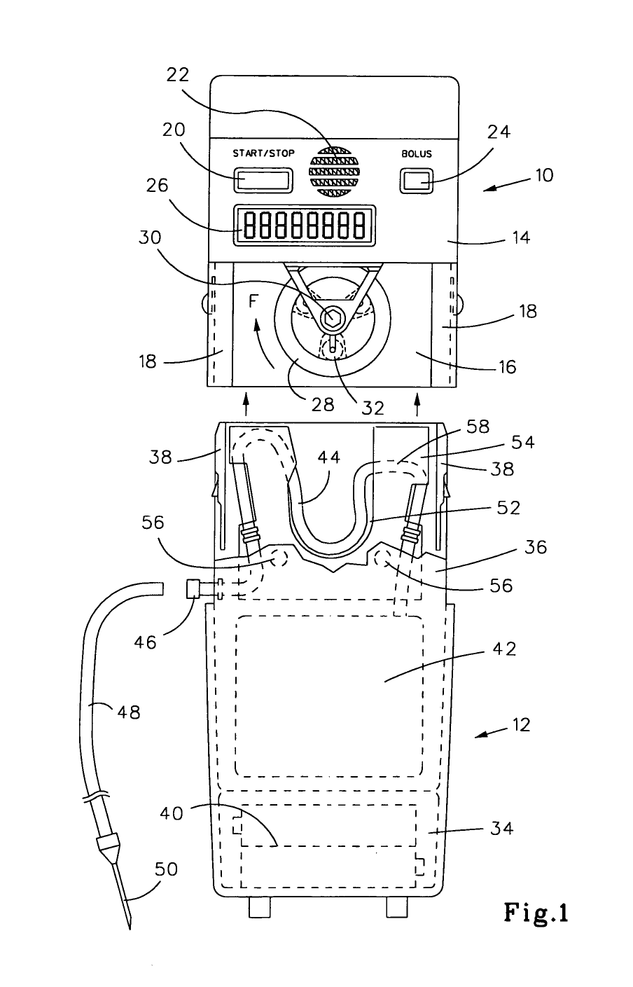

FIG. 1 is an overall view of the pump according to the invention, before coupling the cassette to the pump module;

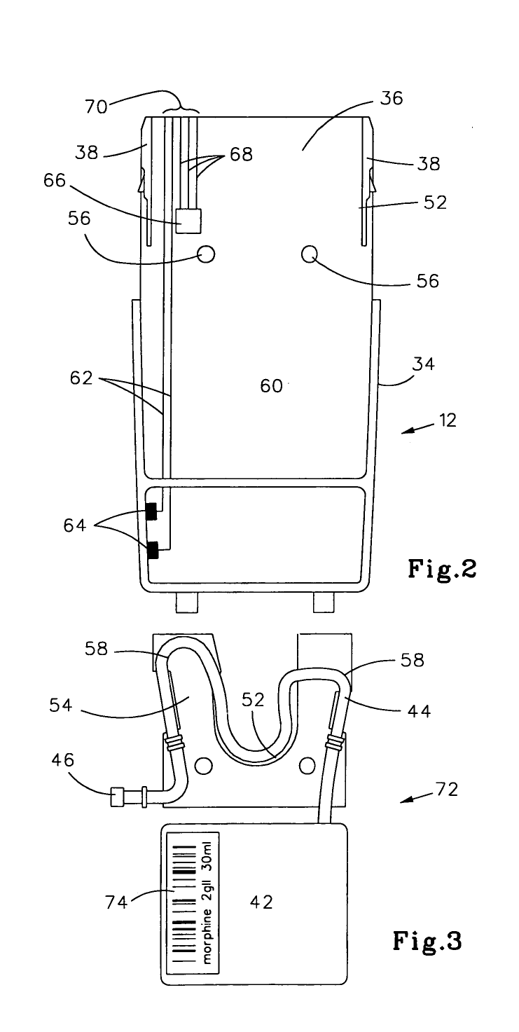

FIG. 2 represents the cassette without its medicament module;

FIG. 3 represents the medicament module; and

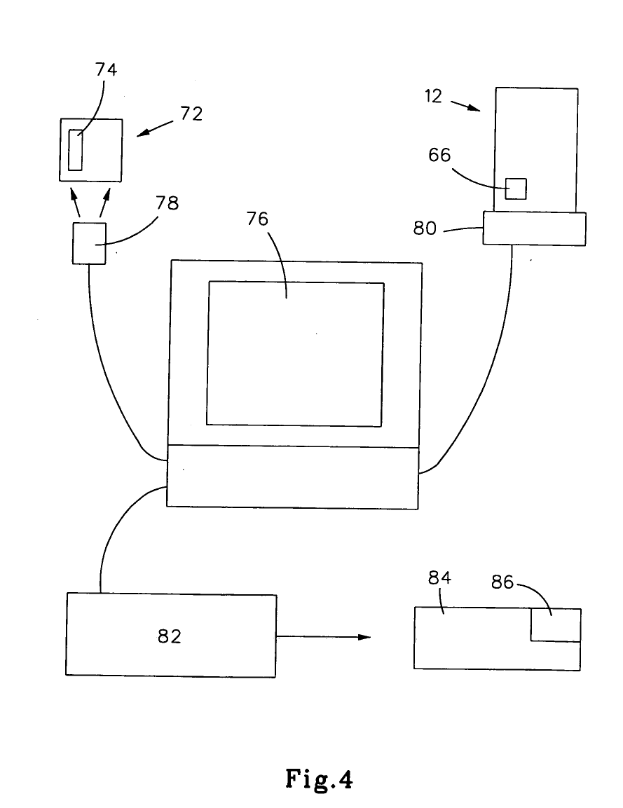

FIG. 4 diagrammatically shows the equipment required for the programming of the pump.

Reference will firstly be made to FIG. 1, which shows a programmable cassette pump according to the invention, made up of a pump module 10 and of a cassette 12 coupled in a detachable manner.

The pump proper is described in great detail in the two previously cited documents. The present description will therefore be limited to its essential elements.

In short, therefore, the pump module 10 comprises a casing 14 of rigid plastic whose cassette side part possesses only a bottom 16 and two parallel lateral slideways 18 serving for the installation of the cassette 12 in the manner of a plug-in.

On its upper face, the casing 14 comprises a START/STOP button 20 serving to control the starting and stopping of the pump, an acoustic warning alarm 22, a BOLUS button 24 serving to trigger the administration of additional doses of medicament, and an LCD display 26.

The casing 14 reveals, between its two slideways 18, a rotor 28 mounted in free rotation about a spindle 30 fixed on the bottom 16 and rotated by means of a stepper motor via a gear train (not visible in the figure). The casing 14 also accommodates a microprocessor in charge of controlling all the abovementioned elements.

The rotor 28 carries three cylindrical idle rollers 32 arranged at 120° to one another and mounted freely in rotation about spindles parallel to the spindle 30.

The cassette 12, shown separated from the pump module 10, comprises a casing 34 of rigid plastic whose pump module side part possesses only an upper face 36 and two parallel lateral arms 38 intended to be inserted into the slideways 18 of the pump module 10. The remainder of the cassette 12 is occupied by two batteries 40 and a plastic pouch 42 filled with a liquid medicament, all arranged under covers.

The pouch 42 is linked to a plastic flexible conduit 44 that sits between the two arms 38 and the end of which emerges outside the cassette where it is closed off by a stopper 46. This stopper will be removed at the time the pump is operated so as to enable a flexible conduit 48 terminating in an injection needle 50 to be plugged in.

In its portion lying between the arms 38, the conduit 44 is applied against a U-shaped rounded bearing piece 52 whose radius is slightly greater than that of the circle described by the outer face of the rollers 32. The piece 52 forms part of a plate 54 made of rigid plastic fixed under the upper face 36 by clipping over two pins 56. It is on this U piece 52 that, once the two parts have been assembled, upon rotation of the rotor 28 in the direction of the arrow F its three idle rollers 32 will, upon each revolution, squeeze the flexible conduit 44 and thus push out, by means of peristaltic motion, the liquid contained in the pouch 42.

On each side of the bearing piece 52, the plate 54 is drilled with a channel 58 in which the conduit 44 sits.

The electrical link between the cassette 12 and the pump module 10 is afforded by conducting tracks (not represented in FIG. 1) deposited on their respective casings.

Reference will now be made to FIG. 2, which shows the cassette 12, viewed from beneath, before installation of the batteries 40, the pouch 42, their cover and the plate 54. Hence, this figure shows the casing 34, with its two arms 38 and the two pins 56 for clipping of the plate 54.

It is thus seen that the bottom 60 of the casing comprises two conducting tracks 62 linking the two terminals 64 for connection of the batteries to the end of the pump module side casing. The bottom 60 also comprises a memory integrated circuit 66, of the type known by the usual term E2PROM, and a second assembly of conducting tracks 68 also linking the memory 66 to the end of the casing, fitted with pads 70, which, upon assembling the cassette 12 with the pump module 10, come into contact with identical pads made on its casing so as to afford interconnection with the microprocessor.

The role played by the memory 66 will now become apparent from the description of the operations for programming and operating the pump according to the invention.

As shown by FIG. 3, one starts with a medicament module 72, represented viewed from beneath, comprising the pouch 42 containing the medicament to be injected, its flexible conduit 44 closed off by the stopper 46, the rigid plate 54 with the bearing piece 52, and the channels 58 in which the conduit sits, all these elements having already been shown in FIG. 1. This assembly, extracted from pharmacy stock, is identified by a label 74 of bar-code type, stuck to the pouch 42 and comprising, encoded and unencoded, the type of medicament and its dosage (morphine 2 g/L) as well as its volume (30 mL).

The only equipment required, shown diagrammatically in FIG. 4, is a computer 76 and, linked to the latter, a bar code reading head 78, a data exchange interface 80 and a printer 82.

The computer 76 contains, in memory, for the medicaments likely to be administered by the pump, the following data:

maximum permitted instantaneous rate,

maximum permitted daily injectable quantity, and

number of boluses authorized per day (which may be 0), maximum authorized volume of each bolus and minimum authorized space between two boluses.

The interface 80, of RS232 type, is furnished with a slot shaped and dimensioned to receive the end of the cassette 12 whose pads 70 come into contact with identical pads, affording interconnection.

The first operation is the reading of the label 74 of the medicament module 72 with the aid of the head 78. The information that it contains (type of medicament and its dosage) triggers the appearance of a screen inviting the user to supply the computer 76 with particulars relating to the patient (name, address, etc.) as well as the instructions relating to the administration of the medicament concerned, within the authorized limits, i.e.:

the injection program: what rates for what durations,

the bolus rate, their authorized number and spacing.

The computer 76 ensures, with each entry, that the values supplied are within the permitted limits contained in its memory. When it is in possession of all these validated elements, it calculates the parameters that must be used by the microprocessor of the pump module 10 in order for the injection of the medicament to be performed in accordance with the instructions given. More precisely, the parameters calculated by the computer 76 are:

for each step of the program corresponding to a constant injection rate, the start and end instants of the step as well as the number of pulses applied to the motor per unit time, this number (which may be 0) determining the speed of rotation of the rotor 30 and hence the injection rate;

for each bolus requested by the patient, its duration and the number of additional driving pulses per unit time.

Once these parameters have been calculated, the empty cassette 12, such as represented in FIG. 2, is inserted into the interface 80. The operator can then order their transfer to the E2PROM memory 66 at the same time as, possibly, the number of boluses authorized per day and their minimum interval.

In order to ensure the safety of the system, the memory 66 retransmits the parameters received to the computer 76, which compares them to the parameters sent, before producing, via the printer 82, a document 84 containing all the information entered into the computer and sent to the memory 66, at the same time as a label 86 with, for example, the name of the patient as well as the duration and the date of the treatment. This label may ultimately be stuck onto the pouch 42.

The operator then merely needs to place the assembly 72, furnished with its label 86, in the now programmed cassette 12. If, as is desirable, the latter comprises a transparent portion at this location, the label will remain visible so as to allow verification at any moment.

Thereafter, the whole can be sent or handed to the carer or to the patient who, in order to operate the pump, needs only to slip the cassette 12 into the pump module 10, remove the stopper 46, plug in the flexible conduit 48 in its place, equipped with its injection needle 50, and engage the pump by pressing the button 20.

Thus, a pump is proposed that is operated solely by way of ultrasimple operations requiring no special training since its programming is afforded automatically by the cassette when it is coupled to the pump module. It is obvious, moreover, that any risk, stemming from poor programming of the pump, at the time it is operated is completely excluded. It should be noted, also, that the procedure for preprogramming the cassette by recording instructions in the E2PROM memory is particularly simple, user-friendly and safe. It will be observed, finally, that this preprogramming, carried out with the aid of a computer and by a qualified professional, who may be the cancer consultant or pain specialist, offers numerous possibilities for choosing administration programs, this being particularly important within these fields.

What is claimed is:

1. A programmable pump for injecting medicaments, comprising two parts that can be coupled in a detachable manner, i.e.:
a cassette (12) containing a liquid to be injected, and
a pump module (10) fitted with actuation means for discharging the liquid from the cassette to the exterior,
characterized in that the cassette comprises an electronic memory (66) in which have previously been recorded data relating to the prescribed injection program and in that, when the two parts are coupled, said actuation means are electrically connected to the memory and respond directly to the data that it contains so as to inject the liquid according to the desired program.

2. The pump as claimed in claim 1, characterized in that the pump module (10) is of peristaltic type and comprises a casing (14) and, inside the latter, a rotor (28) furnished with idle rollers (32), a stepper motor for driving said rotor and a microprocessor for the control of said motor.

3. The pump as claimed in claim 2, characterized in that the cassette (12) comprises a casing (34) in which the memory (66) is fixed and, placed in a detachable manner inside said casing, a medicament module (72) comprising a pouch (42) intended to contain the liquid to be injected, a flexible conduit (44) linking said pouch to the exterior and a bearing piece (52) against which, during the rotation of said rotor, the rollers of the latter will, upon each revolution, squeeze the conduit and thus push out, by means of peristaltic motion, the liquid contained in the pouch.

4. The pump as claimed in claim 3, characterized in that the two casings (14, 34) comprise members for coupling one to the other and are fitted with conducting tracks (62, 68), which, upon the coupling of the casings, are placed in communication in such a way as to interconnect said memory and said microprocessor.

5. The pump as claimed in one of claims 3 and 4, characterized in that the data recorded in said memory (66) comprise, for each step of the program corresponding to a constant injection rate, the start and end instants of the step as well as the number of pulses applied to the motor per unit time, determining the speed of rotation of the rotor and hence the injection rate.

6. The pump as claimed in claim 5, characterized in that the data recorded in said memory comprise, in addition, for each bolus requested by the patient, its duration, the number of additional driving pulses per unit time, the number of boluses authorized per day and their minimum interval.

7. Equipment for programming the pump as claimed in one of claims 3 to 6, characterized in that it comprises:
a computer (76) containing, in memory, for a plurality of medicaments likely to be administered by the pump, the maximum permitted instantaneous injection rate, the maximum permitted daily injectable quantity,
fixed on the pouch (42), a label (74) for identifying the medicament that it contains,
means (78) for reading said label, which are linked to the computer, and
a data exchange interface (80), linked to the computer and designed to link it to the memory of the cassette.

8. The equipment as claimed in claim 7, characterized in that said computer is programmed to perform, on the basis of the information delivered by the label and in response to the instructions supplied by an operator in respect of the desired injection program:
verification that said instructions comply with the permitted values contained in memory,
calculation, for each step of the program corresponding to a constant injection rate, of the start and end instants of the step as well as of the number of pulses applied to the motor per unit time, then
at the request of the operator, transfer of the calculated values to the memory of the cassette.

9. The equipment as claimed in claim 7, characterized in that the computer contains, in addition, for certain, at least, of said medicaments, the number of boluses authorized per day, the maximum volume of each bolus and the minimum space between two boluses.

10. The equipment as claimed in claims 8 and 9, characterized in that the computer is programmed to perform, in addition, the calculation, for each bolus requested by the patient, of its duration and of the number of additional driving pulses per unit time.
